(19) United States
(12) Patent Application Publication
(10) Pub. No.: US 2021/0015549 A1
(43) Pub. Date: Jan. 21, 2021

(54) ABLATION LESION QUALITY

(71) Applicant: Sirona Medical Technologies, Inc., Andover, MA (US)

(72) Inventors: Ali Haghighi-Mood, Andover, MA (US); Richard Jonathan Cohen, Newton, MA (US)

(21) Appl. No.: 16/887,255

(22) Filed: May 29, 2020

Related U.S. Application Data
(60) Provisional application No. 62/854,140, filed on May 29, 2019.

(51) Int. Cl. A61B 18/14

(52) U.S. Cl. CPC A61B 18/1492; A61B 2018/00577

(57) ABSTRACT

Improved ablation procedures and exemplary catheters for use with such procedures are provided. In one example, high quality homogenous lesions with well-defined borders can be created by introducing into a patient body a catheter that includes an electrode that is mounted on a distal end of the catheter, positioning the electrode to be in contact with tissue to be ablated such that at least 65 percent of an exposed distal surface area of the electrode is in contact with the tissue to be ablated, and applying electrical energy to the electrode sufficient to create an ablation lesion in the tissue.

### CROSS-REFERENCE

This application claims the benefit of priority to U.S. Provisional App. No. 62/854,140 of Haghighi-Mood et al., filed on May 29, 2019 and entitled “Improved Ablation Lesion Quality,” which is incorporated herein by reference in its entirety.

### FIELD

Methods and devices are provided for creating lesions in tissue.

### BACKGROUND

Disorders of the electrical functioning of the heart can cause morbidity and mortality. These disorders are generally termed arrhythmias, commonly caused by disorders of impulse formation such as abnormal automaticity of the heart's normal pacemaker and/or disorders of impulse conduction such as partial or complete block of the electrical impulse.

There are various treatments for heart rhythm disturbances, such as with tissue ablation. However, there are many problems and limitations of the currently available methods and devices used for this procedure.

Accordingly, there remains a need for improved ablation procedures.

### SUMMARY

In general, an improved ablation procedure and exemplary catheters for use with such a procedure are provided.

In one embodiment, high quality homogenous lesions with well-defined borders are created by introducing into the body a catheter which includes an electrode which is mounted on a distal end of the catheter, positioning the electrode to be in contact with the tissue to be ablated such that at least 65 percent of an exposed distal surface area of the electrode is in contact with the tissue to be ablated, and applying electrical energy to the electrode sufficient to create an ablation lesion in the tissue. In one embodiment, the electrical energy is radiofrequency energy and is applied at a level between about 4 and about 15 watts for a period of 30 seconds or more. In another embodiment, the radiofrequency energy is applied at a level of about 20 watts or more for a period of about 10 seconds or less. In another embodiment, the catheter contains an irrigation channel and irrigation fluid is delivered at a rate of 6 milliliters/minute or less. In another embodiment, the irrigation fluid flows through a plurality of channels in the electrode. In another embodiment at least fifty percent of the energy that passes out of the catheter through the electrode during ablation of the cardiovascular tissue passes through the tissue to be ablated. In another embodiment, deployable wings at the distal end of the catheter are expanded transversely to the catheter shaft to stabilize the electrode on the tissue surface. Therefore, one embodiment for creating high quality lesions involves expanding the deployable wings located at the distal end of the catheter transversely to the catheter shaft to stabilize the electrode on the tissue surface.

In another embodiment, ablation of cardiovascular tissue may be performed so as to reduce the risk of tissue charring, steam pops and blood coagulation by introducing into the body a catheter that includes an irrigation channel and an electrode that is mounted on a distal end of the catheter, positioning the electrode to be in contact with the tissue to be ablated, delivering fluid through the irrigation channel at a rate of less than or equal to 6 milliliters/minute, utilizing temperature control mode to control the delivery of energy to the electrode, and thereby creating an ablation lesion in the tissue. In another embodiment, at least 65 percent of an exposed distal surface area of the electrode is in contact with the tissue to be ablated. In another embodiment, a thermocouple can be located less than 500 microns from the surface of the ablation electrode is used to monitor temperature. In another embodiment, the energy is radiofrequency energy. In another embodiment, deployable wings are expanded transversely to the catheter shaft to stabilize the electrode on the tissue surface.

In another embodiment, a method can be used for producing high quality lesions and for reducing the risk of charring, steam pops and blood coagulation. The energy delivered to the electrode can be configured to cause irreversible electroporation of the tissue. In one embodiment, this energy is configured to be in the form of pulses wherein the majority of the energy in the pulse is contained in a window of less than 20 milliseconds. In another embodiment, the amplitude of the pulses are between 250 and 2500 volts.

The details of one or more variations of the subject matter described herein are set forth in the accompanying drawings and the description below. Other features and advantages of the subject matter described herein will be apparent from the description and drawings, and from the claims.

### DESCRIPTION OF DRAWINGS

This invention will be more fully understood from the following detailed description taken in conjunction with the accompanying drawings.

FIG. 1 is a cross-sectional, partially-perspective side view of one embodiment of a distal end of a catheter with deployable wings expanded transversely to the catheter shaft.

FIG. 2 is a cross-sectional side view of the distal end of the catheter of FIG. 1 in a linear state with deployable wings collapsed onto the catheter shaft.

FIG. 3A is a partially-transparent and partially-cutaway side view of the catheter of FIG. 1 and FIG. 2.

FIG. 3B is a partially-transparent and partially-cutaway side view of the catheter of FIG. 1 and FIG. 2.

FIG. 3C is a front view of the catheter of FIG. 1 and FIG. 2.

FIG. 3D is a perspective view of the catheter of FIG. 1 and FIG. 2.

FIG. 4A is a table of lesion size and volume generated according to a method provided herein using the catheter system depicted in FIGS. 1-3D compared to lesions generated with the Biosense-Webster ThermoCool cathether system using the standard protocol for use of that catheter to create lesions in tissue. These data were obtained using ex vivo pig tenderloin muscle immerged in a saline bath.

FIG. 4B is a table of lesion size and volume generated according to a method provided herein using the catheter system depicted in FIGS. 1-3D. These data were obtained using ex vivo pig tenderloin muscle immerged in a saline bath. The table compares lesion size obtained when the catheter of FIGS. 1-3D is operated at a higher power (20 watts) and shorter duration (5 seconds) with an irrigation rate of 4 ml/min with the catheter operated at lower power (6-12 watts) and shorter duration (5 seconds) with an irrigation rate of 2 ml/min.

FIG. 5A depicts typical lesions obtained with the catheter system of FIGS. 1-3D and the ThermoCool system.

FIG. 5B is a plot of the reflected light intensity across a typical lesion created with the he catheter system of FIGS. 1-3D and the ThermoCool system.

FIG. 6 illustrates the mechanism how a method provided herein results in more homogeneous sharply defined lesions.

### DETAILED DESCRIPTION

Certain exemplary embodiments will now be described to provide an overall understanding of the principles of the structure, function, manufacture, and use of the devices and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

Further, in the present disclosure, like-named components of the embodiments generally have similar features, and thus within a particular embodiment each feature of each like-named component is not necessarily fully elaborated upon. Additionally, to the extent that linear or circular dimensions are used in the description of the disclosed systems, devices, and methods, such dimensions are not intended to limit the types of shapes that can be used in conjunction with such systems, devices, and methods. A person skilled in the art will recognize that an equivalent to such linear and circular dimensions can easily be determined for any geometric shape. Sizes and shapes of the systems and devices, and the components thereof, can depend at least on the anatomy of the subject in which the systems and devices will be used, the size and shape of components with which the systems and devices will be used, and the methods and procedures in which the systems and devices will be used. Like reference symbols in the various drawings indicate like elements.

Human life depends on the effective functioning of the heart on a second to second basis. If the heart ceases functioning for greater than about four minutes brain death ensues. The pumping function of the heart enables the blood to release carbon dioxide and pick up oxygen in the lungs. The oxygen, along with nutrients, hormones and other needed chemical species are delivered via the circulation to the body's tissues and waste products are removed and eliminated via the kidney and other mechanisms.

The heart can be thought of as an electromechanical four chamber pump. The right and left atria serve as booster pumps that aid in the filling of the heart's main pumping chambers—the right and left ventricles. Venous blood from the body returns through the vena cava to the right atrium. From the right atrium the blood passes into the right ventricle. The right ventricle pumps blood via the pulmonary artery through the lungs. The blood than returns from the lungs through the pulmonary veins, which empty into the left atrium. From the left atrium the blood flows into the left ventricle which then pumps the blood via the aorta into the systemic circulation and then returns to the right atrium through the vena cava thus completing the circuit. Each of the ventricles have inlet and outlet valves to enable the filling of the ventricular chambers when the ventricles are relaxed (diastole) and to ensure the blood is pumped in the forward direction when the ventricles contract (systole).

The mechanical contraction of cardiac muscle is controlled by the propagation of an electrical impulse. The electrical impulse propagates through the heart muscle and initiates the contraction of the muscle. Cardiac muscle cells in the sino-atrial node are specialized to automatically initiate the electrical impulse on a regular basis (normally at a rate of 60 to 100 beats per minute) and thereby serve as the heart's normal pacemaker. Other specialized muscle cells in the atrio-ventricular-node serve to delay conduction of the electrical impulse between the heart's atria and the heart's ventricles. This delay of about 0.1 seconds allows the atria time to contract to aid in the filling of the ventricles before the ventricles begin to contract. Other specialized cells in the ventricles form the His-Purkinje system that serves as a high speed cabling system that rapidly distributes the electrical impulse to the different regions of the ventricles so that the ventricles contract in a synchronous fashion thereby enhancing their ability to function effectively as pumps. If the His-Purkinje system is not functioning for some reason the electrical impulse will propagate through the other ventricular muscle cells but at a slower rate resulting in a less synchronous contraction of the ventricular chambers and a less effective pumping action.

Disorders of the electrical functioning of the heart are important cause of morbidity and mortality. These disorders are generally termed arrhythmias. The proximate cause of approximately one-half of all cardiac deaths are due to arrhythmias. Arrhythmias can be thought as being caused by two general mechanisms.

One mechanism is disorders of impulse formation. One disorder of impulse formation involves abnormal automaticity of the heart's normal pacemaker. For example, failure of the sino-atrial node to form an impulse (sinus arrest), or forming impulses at too slow a rate (sinus bradycardia) or too fast a rate (sinus tachycardia). Abnormal automaticity also applies to the case when other sites in the heart (ectopic sites) begin to function as pacemakers initiating their own electrical impulses. For example, an ectopic site in the atria firing rapidly can generate an arrhythmia called atrial tachycardia and an ectopic site in the ventricles firing rapidly can generate an arrhythmia called ventricular tachycardia.

A second mechanism is disorders of impulse conduction. One type of abnormal conduction is partial or complete block of the electrical impulse. For example, partial block affecting the atrio-ventricular node can abnormally slow impulses passing through the atrio-ventricular node or can result in some of the impulses not propagating through the node at all. Complete atrio-ventricular block is the condition that no impulses propagate through the node. Another type of abnormality of conduction is re-entry. Re-entry involves the circus movement of the electrical impulse through a region of the heart resulting in a self-sustaining pattern of electrical activity. Re-entry can occur in a regular fashion, for example, within a small site within the ventricle. This type of micro-re-entry can be a cause of ventricular tachycardia. Micro-re-entry within the AV node can cause a form of atrial tachycardia. Re-entry can also occur in a random disorderly pattern. Such random re-entry underlies atrial fibrillation and ventricular fibrillation. Atrial fibrillation is characterized by a disorganized pattern of electrical and mechanical activity within the atria. During atrial fibrillation the atria lose their booster pump function in aiding ventricular filling, and just serve as conduits for the blood returning to each of the ventricles. Atrial fibrillation is associated with an irregular and usually an abnormally fast rate of ventricular contraction, less effective filling of the ventricles, and also predisposes to clot formation in the atria. These clots can form emboli that can travel through the circulatory system and cause damage when they lodge in various organs. Emboli that land in the brain can cause strokes. Ventricular fibrillation is characterized by a disorganized pattern of electrical and mechanical activity within the ventricles. While atrial fibrillation is generally not associated with cardiovascular collapse, ventricular fibrillation is. During ventricular fibrillation, the disorganized pattern of mechanical activity in the heart's main pumping chambers results in cessation of blood flow out of the heart, and death unless the ventricular fibrillation is terminated. Clinically ventricular fibrillation can often be terminated by the administration of a large direct current shock to the heart (defibrillation).

There are various treatments for heart rhythm disturbances. Drugs can be used to alter the electrical conduction properties of the heart. Arrhythmias associated with too slow a heart rate (bradyarrhythmias) can often be treated by insertion of a pacemaker, but pacemakers are generally not effective in treating heart rhythm disturbances associated with too rapid heart rates (tachyarrhythmias).

Increasingly, a range of tachyarrhythmias are treated with radiofrequency (RF) ablation. In this technique an electrode tipped catheter is applied to a specific location on the inner (endocardial) or outer (epicaridial) surface of the heart. Then radiofrequency energy is delivered through the electrode to heat the heart tissue near the electrode. The heating ablates the tissue so that It no longer can initiate or conduct electrical impulses. If the site is chosen properly the ablation can prevent the arrhythmia from recurring because the ablated site can either no longer initiate or no longer conduct the electrical impulse responsible for generating the arrhythmia.

While the use of RF ablation is rapidly increasing, there are many problems and limitations of the currently available methods and devices used for this procedure. These problems and limitations greatly limit the effectiveness and safety of RF ablation for the treatment of cardiac arrhythmias.

Complications of the RF ablation procedure itself can include charring of the tissue, perforation of the cardiac wall, creating steam pops that can disrupt the cardiac muscle tissue, coagulation of blood that can create emboli that travel to distant locations in the circulation causing tissue damage; emboli lodging in the brain can cause strokes. Also, RF ablation can cause damage of nearby structures such as the esophagus which lies directly behind the left atrial posterior wall. These complications can cause significant morbidity and also patient mortality.

RF ablation procedures, particularly when performed on the left side of the heart where embolization of coagulated blood or other material to the brain can cause strokes, generally required cooling of the ablation electrode with irrigation fluid delivered through the catheter. This reduces, but does not eliminate, the risk of over-heating the tissue that can cause charring, steam pops, and the coagulation of blood. The irrigation fluid is delivered through the electrode and enters the patient's circulation. During long ablation procedures involves many ablation lesions a large amount of fluid, amounting to several liters, can enter the patients circulation. This large amount of fluid can exacerbate heart failure in patients who have compromised cardiac function.

In order for RF ablation to be effective the site of ablation must be chosen precisely. Lengthy electrical mapping procedures using one or more catheters containing one or more electrical recording electrodes must be utilized in order to precisely locate the site to be ablated. Even when a site is identified, many attempts may be made to ablate the site which fail to successfully block impulse formation, block impulse conduction or otherwise fail to prevent the arrhythmia from recurring or being re-induced while the patient is still lying on the catheterization laboratory. This may result in an unsuccessful procedure, prolonged procedure time, and excessive damage to cardiac tissue.

Often even ablation procedures that appear to successfully abolish the arrhythmia while the patient is lying on the catheterization table, fail in that the arrhythmia recurs at a later time. For example, in a recent study (Packer D L et al, “Effect of Catheter Ablation vs Antiarrhythmic Drug Therapy on Mortality, Stroke, Bleeding, and Cardiac Arrest Among Patients With Atrial Fibrillation”, Journal of the American Medical Society, doi:10.1001/jama.2019.0693 Published online Mar. 15, 2019) 17.1% of patients required a repeat ablation procedure during the first three months after the procedure. During the period starting 3 months after the initial ablation until three years later, 50% of patients had one or more episodes of atrial fibrillation or atrial flutter or atrial tachycardia. Recurrence rates of ventricular tachycardia for RF ablation treatment of ventricular tachycardia are similar (in the range of 30 to 70%) depending on the type of heart disease (Liang J L, et al, “Long-term Outcomes of Ventricular Tachycardia Ablation in Different Types of Structural Heart Disease”, Arrhythmia & Electrophysiology Review 2015; 4(3):177-83). Therefore lack of long term efficacy is a serious problem with current RF ablation procedures.

One of the mechanisms that may account for the high recurrence rate of arrhythmias after RF ablation therapy, is that the RF ablation procedure may damage some of the target tissue but not fully ablate it. Some of the damaged tissue may subsequently heal and recover its ability to form or conduct electrical impulses thus enabling the arrhythmia to recur. One of the mechanisms for this non-complete ablation of targeted tissue is that the ablation procedure may create a spatially non-uniform pattern of damage to the cardiac tissue. In some regions the damage is sufficient to fully ablate the tissue and adjacent regions the tissue is damaged but able to recover and then be able to form or conduct electrical impulses and enabling the arrhythmia to be re-established.

This same mechanism of spatially non-uniform pattern of damage to the tissue this may account for the failure to abolish the arrhythmia in the catheterization laboratory as a non-ablated region of tissue may be either be able to continue to form an electrical impulse or to conduct the electrical impulse around an ablated region. As a result the arrhythmia may not be successfully abolished in the catheterization laboratory.

A methodology currently being explored for ablation of cardiac tissue, but not yet in clinical practice, is irreversible electroporation (also known pulsed field ablation). In this methodology a short duration high voltage pulse, rather than RF energy, is applied to the cardiac tissue (see Fred H. M. Wittkampf, PhD; Vincent J. van Driel, MD; Harry van Wessel, BSc; Kars G. E. J. Neven, MD; Paul F. Grundeman, MD, PhD; Aryan Vink, MD, PhD; Peter Loh, MD, PhD; Pieter A. Doevendans, MD, PhD, Myocardial Lesion Depth With Circular Electroporation Ablation Circulation: Arrhythmia and Electrophysiology 2012; 5:581-586. Also see Reddy V Y, Neuzil P, Koruth J S, Petru J, Funosako M, Cochet H, Sediva L, Chovanec M, Dukkipati S R, Jais P, Pulsed Field Ablation for Pulmonary Vein Isolation in Atrial Fibrillation, Journal of the American College of Cardiology (2019), doi: https://doi.org/10.1016/j.jacc.2019.04.021).

In the method of ablating cardiac tissue by means of irreversible electroporation, a short duration (for example the majority of the energy in the pulse is contained in a window of less than 20 milliseconds duration), high voltage (for example between 250 and 2,500 volts peak amplitude) pulse is administered to the cardiac tissue through an electrode mounted on a catheter. The pulse may be monophasic, biphasic pulse or polyphasic. It is thought that the pulse creates pores in the cell membranes of the cardiac tissue that results in the cells dying via an apoptosis mechanism. Irreversible electroporation of cardiac tissue may also result in local temperature elevation. Wittkampf et al (op cit) report that in their animal studies that “rootlike extensions” were “usually observed at the border of cardiac lesions” that they created by means of electroporation. This may indicate that irreversible electroporation as currently applied may also suffer from spatially nonuniformity of its ablation effect on cardiac tissue. Such nonuniformity might lead either to a failure to abolish impulse formation or conduction at the target site, subsequent recovery of electrical impulse formation or conduction at the target site, or damage to non-targeted tissue.

Therefore, what is needed is an ablation procedure that uniformly and selectively ablates the targeted tissue so that it cannot recover electrical function while at the same time not damaging or ablating nearby tissue and structures that should be preserved. In addition, what is needed is an ablation methodology that reduces the risk of causing tissue charring, steam pops and blood coagulation. Provided herein are ablation procedures that create improved lesions in tissue, especially when treating arrhythmia. As such, an ablation procedure is provided herein that can uniformly and selectively ablate targeted tissue so that the tissue cannot recover electrical function while at the same time not damaging or ablating nearby tissue and structures that should be preserved. In addition, the ablation procedure provided herein can reduce the risk of causing tissue charring, steam pops and blood coagulation.

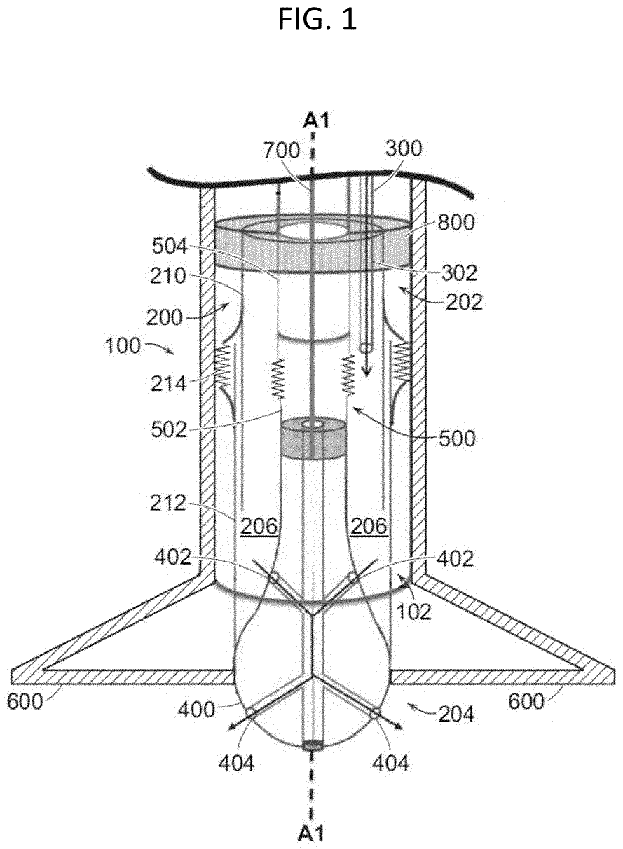

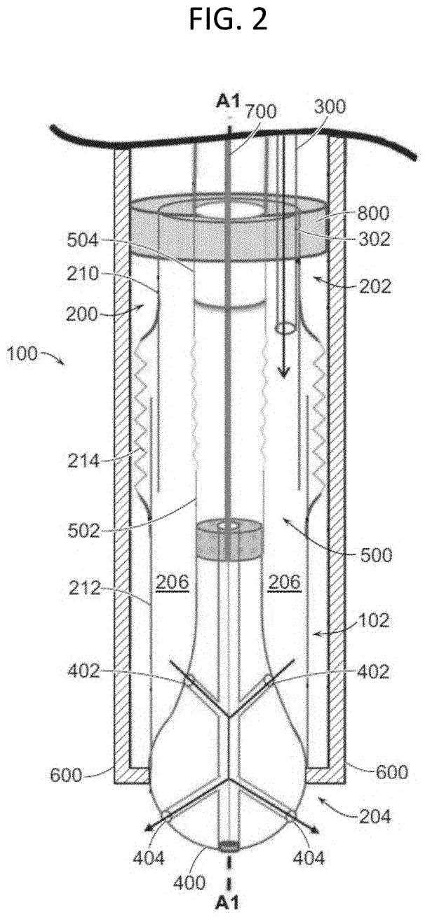

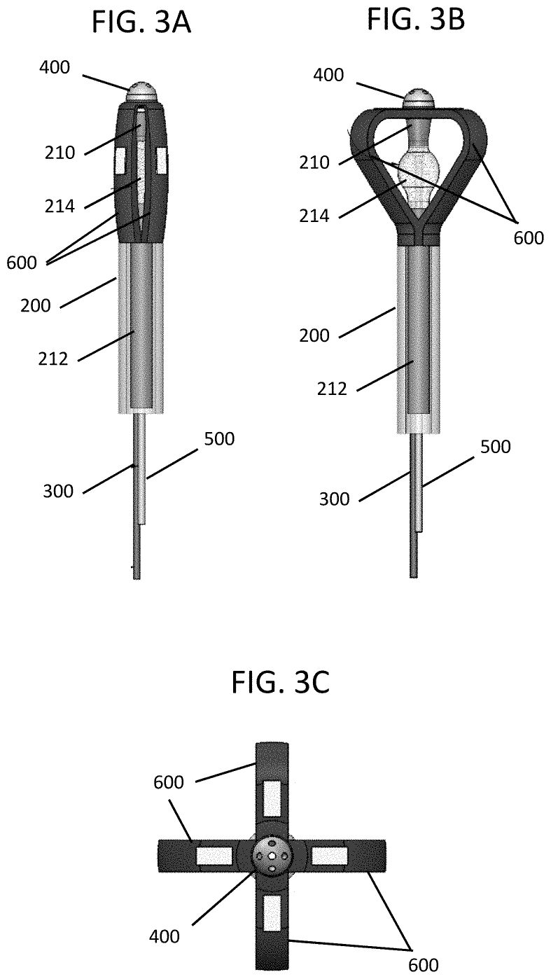

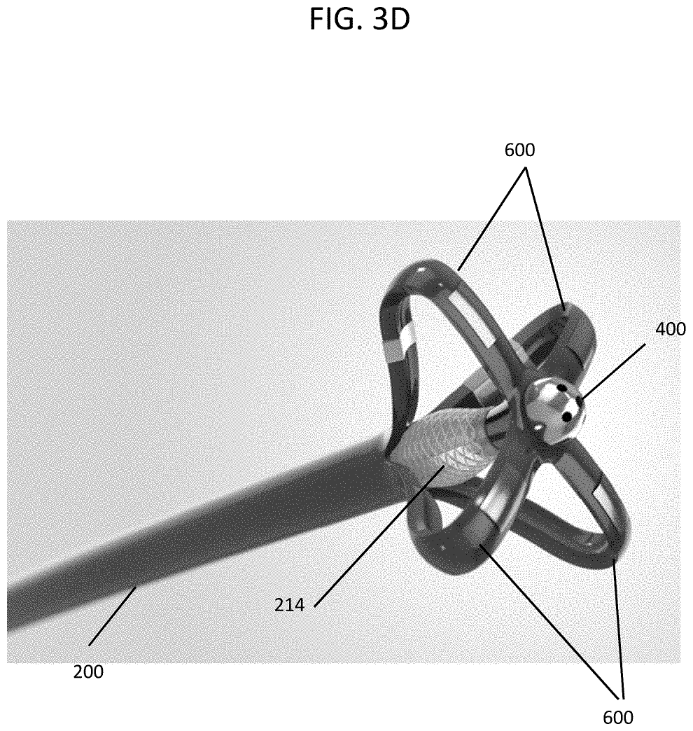

FIGS. 1-3D illustrate one embodiment of a distal end of a catheter with an elongate body 100. The elongate body 100 can be flexible and can have a proximal end, a distal end, and a lumen extending at least partially therethrough. An end effector 102 is disposed at least partially within the distal end of the elongate body 100. The end effector 102 can include an expandable and contractible housing 200 having a fluid inlet 302 at a proximal end and an electrode 400 at a distal end thereof and defining a fluid outlet in the form of at least one pathway formed therethrough. A fluid delivery tube 300 can extend through the elongate body 100 for delivering fluid to the housing 200. The catheter can also include at least one expandable member or wing 600 extending between the distal end of the elongate body 100 and the electrode 400, and an actuator 700 coupled to the electrode for actuating the end effector 102. In this embodiment, proximal movement of the actuator 700 can cause the electrode 400 to retract proximally, thereby causing the expandable member or wing(s) 600 to expand, as shown in FIG. 1, and distal movement of the actuator can cause distal advancement of the electrode body 100, which can cause the expandable member or wing(s) 600 to collapse or compress into a linear configuration, as shown in FIG. 2. For example, the catheter with the elongate body 100 can be maneuvered to a surgical site in the collapsed, linear configuration, as shown in FIGS. 2 and 3A. When at a treatment site, the catheter can either treat tissue in the linear configuration or it can be deployed into the expanded configuration, as shown in FIGS. 1, 3B, and 3C.

The end effector can have a variety of configurations, but as indicated above the end effector is preferably configured to allow advancement and retraction of the electrode using the actuator, while also allowing fluid to be delivered to the electrode. In the illustrated embodiment, the expandable and contractible housing 200 forms a proximal portion of the end effector with the electrode positioned at the distal end thereof. The expandable and contractible housing 200 can have a variety of configurations, but in an exemplary embodiment it is at least partially disposed in the elongate body 100 and at least a portion of it can be configured to move relative to and extend from the distal end of the elongate body 100. The housing 200 can be positioned co-axially with the elongate body 100 such that a longitudinal axis of the housing 200 corresponds to a longitudinal axis A1 of the elongate body 100. In order to define a fluid sealed lumen 206 extending therethrough, a distal end 204 of the housing 200 can be fixed to the electrode 400, and a proximal end 202 of the housing 200 can be sealed by and fixed within the elongate body 100. For example, a heat treatment can be applied to the elongate body to form a substantial end cap 800 on the housing 200, as will be discussed in more detail below. As indicated above, the housing 200 can be configured to expand and contract as the electrode 400 is pulled proximally and pushed distally during use. As illustrated in FIG. 1, the housing 200 can include an upper shaft or tube 210, a lower shaft or tube 212, and an expansion member 214. A proximal end of the expansion member 214 can be attached to the upper shaft 210, and a distal end of the expansion member 214 can be attached to the lower shaft 212. The upper shaft 210 can be fixed at its proximal end to the elongate body 100 and the lower shaft 212 can be fixed to the electrode 400, and one of the upper and lower shafts 210, 212 can be slidably disposed within the other one of the upper and lower shafts 210, 212. As a result, the lower shaft 212 can be configured to slidably move towards and away from the upper shaft 210 in coordination with to movement of the electrode 400. Such a configuration allows the housing 200 to expand and contract. As shown in FIG. 1, a distal end of the upper shaft 210 and a proximal end of the lower shaft 212 overlap one another when the housing 200 is in a deployed state. As shown in FIG. 2, the upper shaft 210 and the lower shaft 212 are moved away from each other in a linear state.

While the upper and lower shafts 210, 212 can be configured to sealing engage one another to prevent fluid leakage from the housing 200, in an exemplary embodiment the expansion member 214 forms a seal around the engagement portion between the two shafts 210, 212. The expansion member 214 can be configured to expand between the two shafts 210, 212 as the shafts 210, 212 slidably move away from each other, and to compress as the shafts 210, 212 move toward one another. The expansion member 214 will thus allow movement of the shafts 210, 212 while maintaining a seal in the fluid sealed lumen 206 within the housing 200 when the housing is in the expanded state or the contracted state. In certain aspects, a length of each of the shafts 210, 212 can be minimized because the expansion member 214 can prevent disengagement between the shafts 210, 212 in the expanded state while still providing a fluid sealed lumen 206 in the housing 200. Because lengths of the shafts 210, 212 can be minimized, an overall length of the housing 200 can be minimized, which allows for a smaller overall end effector length and thus more flexibility of the catheter.

The shafts 210, 212 can be made of stiffer materials relative to the expansion member 214, such as plastics, elastomers, metals, etc., and the expansion member 214 can be made of stretchable and compressible material, such as balloon-like materials, elastomers, plastics, etc. The expansion member 214 can have a variety of forms. For example, the expansion member 214 can be a stretchable, elastic tube. In some situations, a volume of an elastic tube can change when it is stretched, which can result in blood being sucked into the catheter when the catheter configuration is moved between a deployed state and a linear state. This can result in the formation of clots in the fluid compartment. Such clots might subsequently be expelled into the blood stream. Thus it can be helpful to provide support to the elastic tube by providing a support structure, using a more rigid material, etc. The expansion member 214 can be made of a more rigid material that can be folded accordion style. The internal volume of such a tube can experience less change when its length is altered compared to an elastic tube. However, when its length is shortened, the expansion member 214 can potentially fold over on itself, thus impeding fluid flow through the catheter. The expansion member 214 can thus have a central longitudinal support element therein to provide support and prevent incorrect folding and/or collapse. For example, one or both of the shafts 210, 212 can also serve as supports to the expansion member 214 when the housing 200 moves to the contracted state, allowing the expansion member 214 to fold in an orderly controlled manner, similar to an accordion, rather than collapsing in on itself and potentially blocking the fluid sealed lumen 206 of the housing 200. However, the central longitudinal support element is not limited to one or both of the shafts 210, 212. For example, the central longitudinal support element can include one or more other catheter elements, such as the actuator 700, a wire connected to a temperature sensor (such as a thermocouple) located in a tip of the catheter, a tube containing the actuator and/or thermocouple wire, etc. Because the stretchable tube is prevented from folding over on itself, fluid flow through the catheter in general and the expansion member 214 in particular is not impeded by such folding. In addition, in embodiments where the central longitudinal support element is a tube containing other catheter elements, such as the actuator wire or the wire connected to a thermocouple, these other elements can be isolated from the irrigation fluid and thus any adverse effects on the function of these elements that would result from contact with the irrigation fluid can be minimized. In some embodiments, a portion of the central longitudinal support element itself can include a stretchable element that can serve to reduce or prevent leaks around the actuator wire and/or thermocouple wire. A variety of other supports can be used with the expansion member 214, such as tubes, braces, stiffer materials, pre-formed or folded material that will maintain a more rigid accordion fold when contracted, etc. The fluid sealed lumen 206 can be configured to allow delivery of fluid to the electrode 400 with more consistent flow rates and more ideal fluid pressures, thus helping to provide a smoother function of the device and better irrigation and ablation of tissue.

As indicated above, the proximal end 202 of the housing 200 can be coupled to the elongate body 100, and can be fluidly sealed to prevent any fluid from moving proximally from the fluid sealed lumen 206 of the housing into other proximal parts of the catheter. The proximal end 202 of the housing 200 can be coupled to the elongate body 100 through a variety of means. For example, the elongate body 100 can be heat treated, causing the elongate body 100 to melt around and across an open proximal end of the housing 200, as well as around other components extending therethrough, such as the fluid delivery tube 300 and a sealing shaft 500 that receives the actuator. The process of heating can seal the proximal end 202 of the housing 200, as shown in FIG. 1, forming a cap 800 of melted material and achieving fixation and a fluid seal through one process. A variety of other means for both fixation and sealing can be used, however. For example, the proximal end 202 of the housing 200 can be sealed using a cover, a separate and distinct cap, seal, etc., placed across the opening of the proximal end 202 of the housing 200. An end cap or cover can also be formed as part of the upper tube.

As indicated above, the housing 200 is configured to receive fluid therein and to direct fluid to the electrode. Fluid can be introduced to the housing 200 through a variety of means, such as via a fluid delivery tube 300 extending through an inlet formed in the sealed proximal end of the housing 200. The fluid delivery tube 300 can be configured to deliver fluid through the catheter and into the fluid sealed lumen 206 of the housing 200. The fluid delivery tube 300 can at least partially extend through the catheter and the elongate body 100, for example extending from a proximal end of the catheter and terminating in the fluid sealed lumen 206 of the housing 200. The cap 800 can be formed after the fluid delivery tube 300 is in place, thus sealing the proximal end 202 of the housing 200 and securing the fluid delivery tube 300 in place. However, the fluid delivery tube 300 can be configured to pass through a variety of covers over the proximal end 202 of the housing 200 and can be secured in place through a variety of means, such as by use of adhesive or pins. The fluid delivery tube 300 can be configured to deliver a consistent flow of fluid into the fluid sealed lumen 206.

The electrode 400 can be positioned at a distal end of the elongate body 100, and it can be configured to move between a proximally deployed or vector position, as illustrated in FIG. 1, to an advanced or linear position, as illustrated in FIG. 2. In the retracted position, the electrode 400 can be at least partially retracted into the elongate body 100, and in the expanded position, the electrode 400 can extend distally away from the distal-most end of the elongate body 100. A proximal end of the electrode 400 can be at least partially disposed in the distal end 204 of the expandable and contractible housing 200, expandable along its longitudinal dimension such that it is stretchable. For example, the lower shaft 212 of the housing 200 can attach to the electrode 400 such that a fluid seal can be formed along the engagement of the electrode 400 and the lower shaft 212. This engagement can seal the distal end of the fluid sealed lumen 206 of the housing 200. The electrode 400 can be configured to be moved distally and proximally, which can cause the housing 200 to expand and contract as the lower shaft 212 moves distally and proximally with the electrode 400. The electrode 400 can have one or more fluid paths therethrough configured to allow fluid flow from the fluid sealing lumen 206 to a position distally external from the entire catheter to reach tissue to be irrigated and/or ablated. For example, the electrode 400 can have one or more inlet ports 402 on a proximal half thereof that open inside of the fluid sealed lumen 206 of the housing 200 and connect via one or more fluid channels within the electrode 400 to outlet ports 404 on a distal half thereof that open outside of the catheter entirely. The electrode 400 can thus be configured to receive fluid through the inlet ports 402 from the fluid sealed lumen 206 of the housing 200 and can be configured to expel the fluid from the outlet ports 404 to tissue that is distally positioned in front of the electrode 400. Because the electrode 400 can be sealed to the housing 200 at the distal end of the fluid sealed lumen 206 and because the housing 200 can expand and contract with the electrode 400, irrigation can be performed when the electrode 400 is retracted or advanced (e.g. in either of the deployed or linear states shown in FIGS. 1 and 2).

In order to move the electrode, the end effector 102 can be actuated through a variety of means, such as by use of the actuator 700 illustrated in FIG. 1. The actuator 700 can have a variety of forms, such as one or more wires and/or cables. The actuator 700 can extend through the elongate body 100 between a proximal end of the catheter and the end effector 102. As illustrated in FIG. 1, the actuator 700 can extend through the lumen 206 of the housing 200 and can be fixed to a proximal end of the electrode 400. The actuator 700 can be slidable relative to the elongate body 100 such that the actuator 700 can slide distally and proximally while the elongate body 200 remains unmoved relative to the actuator 700. The actuator 700 can be in the form of a wire that is rigid enough to push the electrode 400 distally into the extended state illustrated in FIG. 2, and strong enough to pull the electrode 400 proximally into the retracted state illustrated in FIG. 1, while still being flexible enough to extend through bending and angled sections of the catheter. The actuator 700 can include an electrically-conductive wire that can deliver energy to the electrode 400 during tissue ablation. The actuator 700 can also have one or more coatings thereon to protect surrounding components from the electrical energy deliverable through the actuator 700 and to protect the actuator 700 from its surrounding environments.

In order to allow the actuator to couple to the electrode, the actuator 700 can be co-axial with the housing 200. The actuator 700 can be configured to extend through the proximal end 202 of the housing 200 and through the fluid sealed lumen 206 to engage with the proximal end of the electrode 400 such that the actuator 700 is slidable relative to the proximal end 202 of the housing 200 while the fluid seal of the fluid sealed lumen 206 is maintained. The fluid seal of the fluid sealed lumen 206 can be maintained even with the slidable actuator 700 disposed therein through a variety of means. For example, a sealing shaft 500 can extend around at least a portion of the actuator 700 and can be configured to create a fluid barrier between the actuator 700 and fluid in the end effector 102, such as the fluid sealed lumen 206. The sealing shaft 500 can extend at least partially through the end effector 102, for example extending from the electrode 400, through the fluid sealed lumen 206 of the housing 200, to the proximal end 202 of the housing 200, and optionally into the proximal part of the catheter. The actuator 700 can extend through a lumen within the sealing shaft 500, and the sealing shaft 500 can thus prevent the fluid in the fluid sealed lumen 206 of the housing 200 from contacting the actuator 700. For example, a distal end of the sealing shaft 700 can be sealably fixed to the proximal end of the electrode 400, and a proximal end of the sealing shaft 500 can extend into and optionally through the proximal end 202 of the housing. The sealing shaft 500 can be fixed in place relative to the proximal end 202 of the housing 200. For example, when the cap 800 is formed, the sealing shaft 500 can be positioned before formation and fixed in place relative to the proximal end 202 of the housing during cap formation. However, the sealing shaft 500 can also be fixed in place through a variety of other means, such as adhesives, pins, engagement with other seals, caps, or covers added to the proximal end 202 of the housing 200, etc.

The sealing shaft 500 can be configured to expand and contract with the housing 200 as the electrode 400 is moved distally and proximally. Because the sealing shaft 500 is able to expand and contract with movement of the electrode 400, the sealing shaft 500 can be configured to provide a sealed passage for the actuator 700 through the fluid sealed lumen 206 of the housing 200, which can protect the actuator 700 and can allow the lumen 206 in the housing 200 to remain fluid sealed even as the actuator 700 is moved back and forth through the proximal end 202 of the housing 200. Without the sealing shaft 500, pressure from the fluid flowing into the housing 200 could cause fluid to flow through the opening in the proximal end 202 of the housing 200 around the actuator 700, and into the rest of the catheter. The required movement of the actuator 700 through the proximal end 202 of the housing 200 makes fluidly sealing the proximal end 202 through other means, such as by use of O-rings, difficult to achieve and consistently maintain. In particular, O-rings or other seals will create friction, thereby preventing movement of the actuator. Accordingly, the sealing shaft 500 allows for free movement of the actuator 700, while fluidly separating the actuator 700 from the fluid sealed lumen 206 of the housing 200, thus allowing fluid to be delivered directly to the electrode.

The sealing shaft 500 can include a flexible sealing portion that is configured to expand and contract with movement of the actuator 700 and the electrode 400. In the illustrated embodiment, a sealing member 502 forms a distal portion of the sealing shaft 500 and is sealed on its distal end to the proximal end of the electrode 400. The proximal end of the sealing member 502 can be sealed to a rigid portion 504 of the sealing shaft 500 that can extend through the proximal end 202 of the housing 200. However, the sealing member 502 can also be sealed directly to the proximal end 202 of the housing 200 and/or a cap, cover, seal, etc. that is used to close the proximal end 202. Alternatively, the sealing member 502 can be integral and unitary with the sealing shaft 500.

When the actuator 700 is moved proximally and distally to move the electrode 400 between the contracted and the expanded states, the sealing member 502 is configured to stretch and contract with movement of the actuator 700 so that the electrode 400 can be moved without breaking the fluid barrier between the fluid sealed lumen 206 of the housing 200, the proximal end 202 of the housing 200, and the actuator 700. The sealing member 502 can be made from any material that can expand and contract, such as various elastomers, plastics, elastics, balloon-like materials, etc. The sealing shaft 500 can also have a rigid portion 504 that extends through the proximal end 202 of the housing 200 and that is configured to be secured in place by the cap 800. The rigid portion 504 can be made of a variety of materials that are configured to withstand the heat treatment applied to the elongate body, such as various plastics or metals.

As indicated above, the catheter also includes at least one expandable member extending between the electrode and the distal end of the elongate body. In an exemplary embodiment, the catheter can include four expandable members positioned equidistant there around. As the electrode 400 advances and retracts, the expandable members 600 that extends between the distal end of the elongate body 100 and the electrode 400 moves between an initial linear configuration for advancement through a body lumen, to flared or expanded configuration. In the deployed configuration, the expandable members can bend around a midpoint there along to extend substantially perpendicular relative to the elongate body 100, forming a flower pedal shape or propeller blade shape around the electrode 400. The one or more expandable members 600 can be configured to extend longitudinally relative to the elongate body 100 when the electrode 400 is extended distally away from the elongate body 100. As the electrode 400 and the distal end 204 of the housing 200 extend distally, the expandable members 600 can be configured to flatten against an exterior surface of the housing 200 as the distal end of the catheter takes on a linear shape. The distal ends of the one or more expandable members 600 can couple to the electrode 400 at a point between the proximal and distal end of the electrode 400, such as at a point corresponding to the engagement between the distal end 204 of the housing 200 and the electrode 400.

The expandable members 600 can each have one or more electrodes disposed thereon, such as for recording electrical signals, and positioned on a distal portion of the expandable member 600 such that each electrode is configured to be approximately perpendicular to the elongate body 100 when the electrode 400 is retracted in the contracted state and each expandable member 600 is in a flared or winged state. The one or more electrodes on the expandable members 600 can be configured to operate in coordination with electrode 400 to provide ablation to a larger surface area of tissue than just the electrode 400 alone. Additional details concerning the catheter generally and the interaction between the expandable member(s) and a central electrode are discussed in detail in U.S. Pat. No. 8,882,761, filed Jul. 15, 2008, U.S. Pat. No. 9,717,558, filed Nov. 7, 2014, and patent application Ser. No. 15/661,606, filed Jul. 27, 2017, all of which are hereby incorporated by reference herein in their entireties. The electrode(s) on the one or more expandable members 600 can be coupled to and receive energy from the actuator 700.

In use, the end effector 102 can be arranged in the linear state as shown in FIG. 2 and the catheter can be advanced through a body lumen of a patient to position the end effector 102 at a surgical site with tissue to be treated, such as tissue requiring ablation and/or irrigation. The actuator 700 can be proximally retracted to proximally retract the electrode 400, causing the end effector 102 to move to the deployed state, as illustrated in FIG. 1, with the expandable members in the expanded configuration. As the electrode 400 is retracted, the housing 200 compresses and reduces in length as the shafts 210, 212 move towards each other and overlap with one another and the expansion member 214 contracts before folding over one or both of the shafts 210, 212 as it compresses entirely. The sealing member 502 of the sealing shaft 500 can also begin compressing and folding together as the actuator 700 retracts the electrode. The catheter can be manipulated to position the electrode 400 and one or more of the expandable members 600 in contact with tissue to be treated. The catheter can be actuated to deliver energy to the electrode 400 and any electrodes arranged on the expandable members 600 and/or to deliver fluid through at least one fluid pathway in the electrode 400. The fluid can flow through the fluid sealed lumen 206 and through the ports 402, 404 in the electrode. When ablation and/or irrigation is finished, the actuator 700 can be pushed distally to cause the end effector 102 to return to the linear state. The housing 200 can expand in length as the shafts 210, 212 move away from each other and the expansion member 214 expands to keep the shafts 210, 212 engaged with each other while allowing the shafts 210, 212 to move away. The sealing member 502 of the sealing shaft 500 also can unfold and expand as the electrode 400 moves distally. The catheter can be maneuvered to another site or removed from the patient. Because of the expandable and contractible fluid channel in the end effector 102, fluid can be successfully delivered to tissue with the end effector 102 in the expanded state, allowing ablation with fluid and/or irrigation to be performed successfully in the expanded state. The catheter(s) disclosed herein can be steered through a variety of means, which are well-known in the art.

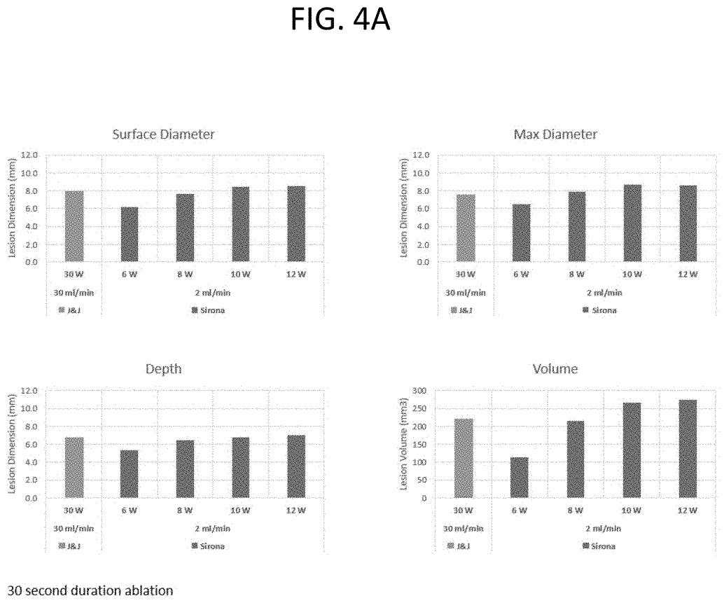

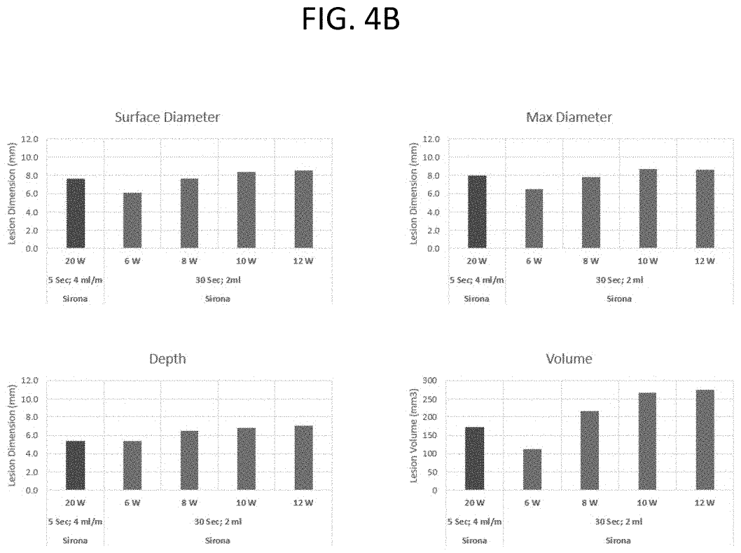

As such, FIGS. 3A-3D illustrate an irrigated ablation catheter that has a shaft containing an irrigation tube to deliver irrigation fluid to a central ablation electrode located on the tip of the shaft. There are four deployable wings on the catheter each containing a recording electrode. The deployable wings may deployed in a longitudinally expanded mode or a longitudinally compressed mode. In the longitudinally expanded mode, the deployable wings are collapsed along the shaft of the catheter and in the longitudinally compressed mode the deployable wings are expanded transversely to the catheter shaft and surround the central ablation electrode. An actuator cable which passes through the center tube is used to move the catheter between longitudinally expanded and longitudinally compressed modes. The irrigation fluid passes from the irrigation tube through an expandable sheath to enable irrigation of the central electrode in either longitudinally expanded or compressed modes. Wires passed through the central tube connect to the central ablation electrode and each of the recording wing electrodes. Also, a wire pair is connected to a thermocouple located in the central ablation electrode. FIG. 4A illustrates a table of lesion size and volume generated according to the method of this invention using the catheter system depicted in FIGS. 1-3D, such as the Sirona Medical Technologies (SMT), compared to lesions generated with the Biosense-Webster ThermoCool cathether system using the standard protocol for use of that catheter to create lesions in tissue. These data were obtained using ex vivo pig tenderloin muscle immerged in a saline bath. FIG. 4B illustrates a table of lesion size and volume generated according to method provided herein using catheter system depicted in FIGS. 1-3D. These data were obtained using ex vivo pig tenderloin muscle immerged in a saline bath. The table compares lesion size obtained when the SMT catheter is operated at a higher power (20 watts) and shorter duration (5 seconds) with an irrigation rate of 4 ml/min with the SMT catheter operated at lower power (6-12 watts) and shorter duration (5 seconds) with an irrigation rate of 2 ml/min.

While an exemplary catheter is provided above, the ablation procedure provided herein can be implemented using a variety of catheters, such as those described in US Patent Application Publication No. 2019/0090942 entitled “Catheter and Method for Improved Irrigation” and filed on Sep. 24, 2018, which is hereby incorporated by reference in its entirety.

In one embodiment of this invention, a lesion in a biological tissue is created by positioning an electrode mounted on a distal end of a catheter to be in contact with biological tissue to be ablated such that at least 65 percent of the distal exposed surface area of the electrode illustrated in FIGS. 1-3D is in contact with the biological tissue to be ablated, the electrode is irrigated with an irrigation fluid delivered through the catheter, and radiofrequency (RF) energy to the electrode in order to ablate the cardiovascular tissue. In particular, one may position the central ablation electrode of this catheter to be placed in contact with biological tissue so at least 65% of the distal exposed distal surface of the ablation electrode is in contact with the tissue.

FIG. 4A shows data obtained creating lesions according to this method in ex vivo pig tenderloin muscle immerged in a saline bath. These data were obtained using the Sirona Medical Technologies (SMT) catheter described in FIGS. 1-3D compared to the lesion dimensions and volume obtained using the Biosense-Webster ThermoCool Catheter which is the most widely using ablation catheter used in cardiac electrophysiology applications. Here we see that the SMT catheter created the same size lesions using 6-12 watts of RF power and 2 milliliters/min of irrigation fluid compared to the standard settings of 30 watts of RF power and 30 milliliters per minute irrigation fluid using the ThermoCool catheter. That is, the same size lesions were obtained with the SMT catheter using approximately 66% less power and approximately 90% less irrigation fluid.

FIG. 4B shows that the SMT catheter can achieve similar size lesions when operating at a higher power (20 watts) and shorter duration (5 seconds) with an irrigation rate of 4 ml/min as when the SMT catheter operates at lower power (6-12 watts) and shorter duration (5 seconds) with an irrigation rate of 2 ml/min.

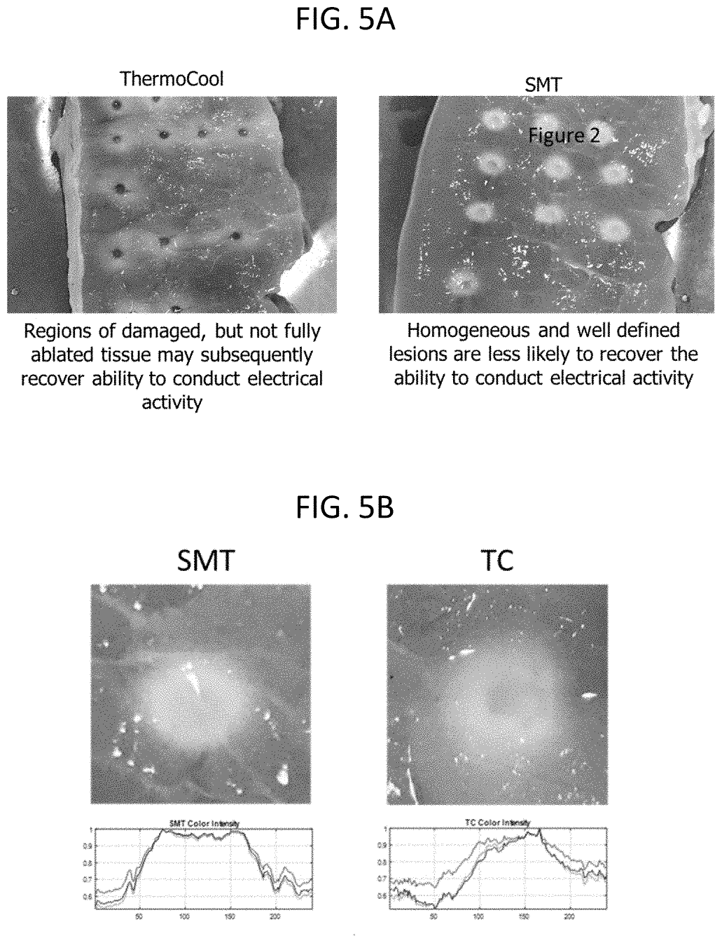

In addition to achieving the same size lesions as obtained with the ThermoCool catheter when using the SMT catheter with far lower power and irrigation rate, in the course of conducting these experiments it was discovered that the quality of the lesions obtained with the SMT catheter was far superior as shown in FIG. 5A. The lesions obtained with the SMT catheter were homogenous and had sharply defined borders whereas the lesions obtained with the ThermoCool catheter were inhomogeneous and had poorly defined borders. The plot of reflected light intensity across a typical lesion in FIG. 5B illustrates this point. For the lesion created with the SMT catheter, as one proceeds from left to right along the horizontal axis of the plot, the reflected light intensity rises sharply at the left hand border of the lesion, is flat across the lesion itself, and then drops sharply at the right hand border of the lesion. In contrast. for the lesion created with the ThermoCool catheter, as one proceeds from left to right along the horizontal axis of the plot, the reflected light intensity rises gradually at the left hand border of the lesion, never achieves a plateau, and then drops gradually at the right hand border of the lesion. In addition, most of the lesions created with the ThermoCool catheter evidenced a region of tissue char at the center of lesion underneath the location where the ablation electrode had been placed.

The above results regarding the quality of the lesions were unexpected, novel and are of utmost clinical significance. One of the problems with RF ablation therapy for cardiac arrhythmias is that often the arrhythmia recurs some time after the ablation procedure has been performed. One mechanism for this recurrence is that some of the cardiovascular tissue is damaged but not fully ablated. The data presented above demonstrate that the lesions obtained with a standard ablation catheter are in fact highly heterogeneous indicating that some regions may be fully ablated and other regions not. If the regions that are not fully ablated recover electrical function they may once again be able to either form electrical impulses or conduct electrical impulses allowing the arrhythmia to recur. In contrast, the lesions obtained with the SMT catheter were highly homogeneous and well defined, indicating that the region of the lesion is fully ablated while adjoining tissue is not. The fact that the edges of the lesions created with the SMT catheter are well defined is also of great clinical significance because one of the complications of ablation therapy is damaging or ablating adjacent tissue that one does not want to harm and is important for normal function.

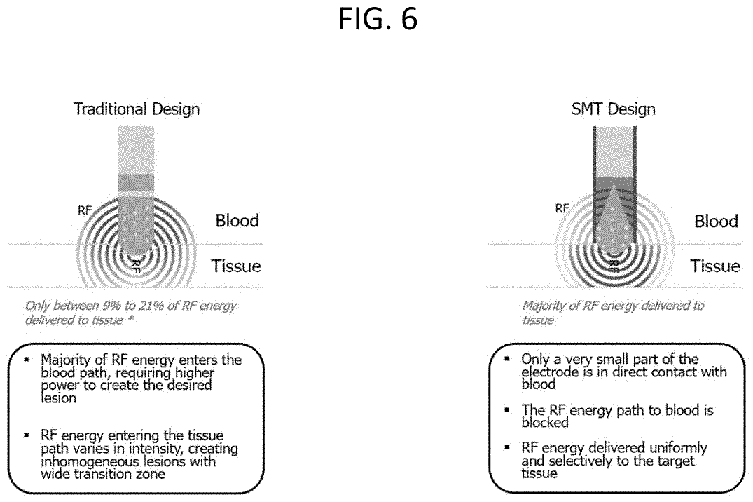

The inventors analyzed why these surprising and unexpected results were obtained. FIG. 6 demonstrates the results of this analysis. With the ThermoCool catheter, as is the case with other standard catheters, the large majority of the RF energy travels through the blood and only a small minority of the energy enters the target tissue. The blood is moving and is continuously stirred by the large volume of irrigation fluid coming out of the top and sides of the ablation electrode. The electrical conductivities of the irrigation fluid, the blood and the tissue are not the same. The RF energy may reach the tissue through various pathways—for example, directly from the ablation electrode to the tissue or through the irrigation fluid and blood and then to the tissue. The intensity of the RF field experienced by the tissue may be highly heterogeneous depending on the pathways taken by the RF energy through different media with different conductivities. Similarly, the temperatures of the irrigation fluid, blood and tissue are all different and vary in space and in time. Thus the cooling effect of the continuously stirred irrigation fluid and blood on the tissue will also be spatially inhomogeneous. The result is a very non-uniform heating of the tissue. In contrast, with the SMT catheter the great majority of the exposed surface area of the electrode is contact with tissue and there is very little contact with blood. Moreover, the irrigation fluid flows at a much slower rate and more evenly flushes the surface of the electrode with a thin film of fluid. The RF field predominantly flows from the exposed surface area of the electrode through the thin film of irrigation fluid directly into the tissue. As a result the RF field and cooling effect of the irrigation fluid is much more spatially uniform and unchanging in time. The result is a much more homogeneous heating and ablation of the tissue resulting in a well defined lesion.

Therefore, in one embodiment high quality homogenous lesions with well defined borders are created by introducing into the body a catheter which includes an electrode which is mounted on a distal end of the catheter, positioning the electrode to be in contact with the tissue to be ablated such that at least 65 percent of an exposed distal surface area of the electrode is in contact with the tissue to be ablated, and applying electrical energy to the electrode sufficient to create an ablation lesion in the tissue. In another embodiment the electrical energy is radiofrequency energy and is applied at a level between about 4 and about 15 watts for a period of 30 seconds or more. In another embodiment, the radiofrequency energy is applied at a level of about 20 watts or more for a period of about 10 seconds or less. In another embodiment, the catheter contains an irrigation channel and irrigation fluid is delivered at a rate of 6 milliliters/minute or less. In another embodiment the irrigation fluid flows through a plurality of channels in the electrode. In another embodiment at least fifty percent of the energy that passes out of the catheter through the electrode during ablation of the cardiovascular tissue passes through the tissue to be ablated. In another embodiment deployable wings at the distal end of the catheter are expanded transversely to the catheter shaft to stabilize the electrode on the tissue surface.

In addition, with standard ablation catheters such as the ThermoCool, the during cardiac ablation the heart is moving with each heart beat and as a result the electrode tip can slip on the surface of the cardiovascular tissue and can also slip off the tissue altogether during the course of the ablation procedure. This motion of the ablation electrode with respect to the tissue will of course create spatial variation in the heating that results and thus contribute to the inhomogeneity of the lesions created. The SMT catheter has deployable wings, which can stabilize the contact of the central ablation electrode with the tissue and prevent it from slipping. Deploying these wings so they are expanded transversely to the catheter shaft during ablation procedures, will enable the SMT catheter to form well defined lesions and to not inadvertently damage adjacent tissue that one does not wish to harm. Therefore, one embodiment for creating high quality lesions involves expanding the deployable wings located at the distal end of the catheter transversely to the catheter shaft to stabilize the electrode on the tissue surface.

In addition, in the course of conducting these experiments it was found that with the SMT catheter there was no charring of the tissue nor steam pops in tissue when using 10 watts or less of RF power and 2 ml/min or more of irrigation, whereas there were multiple instances of tissue charring and steam pops obtained when using the ThermoCool catheter with its standard settings of 30 watts of power and 30 ml/min of irrigation. Also, when the SMT catheter was operated at higher power (20 watts) and shorter duration (5 seconds) with an irrigation rate of 4 ml/min (see FIG. 5B) no charring of tissue nor steam pops were observed. Charring and steam pops occurring during cardiovascular ablation procedures can disrupt tissue integrity, create material that may embolize causing remote tissue damage and strokes.

RF ablation of cardiac tissue may be conducted using the SMT catheter in temperature control mode while using a low irrigation flow rate of 2 milliliters/minute per minute. In temperature control mode the delivery of RF energy to the catheter is modulated to maintain below a threshold value the temperature monitored by a thermocouple located inside the ablation electrode. The threshold value is chosen to prevent charring of tissue, steam pops and blood coagulation. The low irrigation rate employed with the SMT catheter results in the temperature at the surface of the ablation electrode accurately reflecting the temperature of the adjacent tissue. In addition, the thermocouple (aligned on axis A1 in FIGS. 1 and 2) located is located very close (between 127-380 microns) to the surface of the ablation electrode. This close location of the thermocouple to the surface of the ablation electrode also promotes an accurate reading of the adjacent tissue temperature. Therefore, when using the SMT catheter in temperature control mode with low irrigation flow rate the tissue temperature can be accurately monitored by the thermocouple and thereby the risk of tissue charring, steam pops, and blood coagulation can be minimized.

In contrast conventional catheters, such as the Biosense-Webster ThermoCool catheter typically require irrigation rates of 30 ml/min or more and the thermocouple may not be placed as close to the surface. Therefore, with these conventional catheters the temperature measured by the thermocouple does not accurately reflect the temperature of the adjacent tissue. As a result, the use of temperature control mode cannot be used as effectively to prevent tissue charring, steam pops, and blood coagulation.

Therefore in one embodiment, ablation of cardiovascular tissue may be performed so as to reduce the risk of tissue charring, steam pops and blood coagulation by introducing into the body a catheter which includes an irrigation channel and an electrode which is mounted on a distal end of the catheter, positioning the electrode to be in contact with the tissue to be ablated, delivering fluid through the irrigation channel at a rate of less than or equal to 6 milliliters/minute, utilizing temperature control mode to control the delivery of energy to the electrode, and thereby creating an ablation lesion in the tissue. In another embodiment at least 65 percent of an exposed distal surface area of the electrode is in contact with the tissue to be ablated. In another embodiment, a thermocouple located less than 500 microns from the surface of the ablation electrode is used to monitor the temperature. In another embodiment the energy is radiofrequency energy. In another embodiment, deployable wings are expanded transversely to the catheter shaft to stabilize the electrode on the tissue surface.

The SMT catheter depicted in FIGS. 1-3D can be coupled with a power source that generates short duration high voltage pulses suitable for ablating tissue by means of irreversible electroporation instead of being coupled with an RF power source. There is evidence that irreversible electroporation when used with standard catheter systems will also generate spatially inhomogeneous damage to tissue resulting in lesions that may either not abolish the arrhythmia in the catheterization laboratory or lead to recurrence of arrhythmia subsequently. The method of this invention as described above may be applied equally well when the power source is adapted to ablation by means of irreversible electroporation rather than ablation by means of applying radiofrequency power and the advantages of using the SMT catheter apply equally well when irreversible electroporation is used to ablate the tissue. In particular, the direct contact of the ablation electrode with the tissue and the minimal amount of energy that passes through the blood path will enhance uniformity of the resulting tissue lesion. This in turn will lead to more effective ablation improving the ability to terminate the arrhythmia in the catheterization laboratory and reducing the re-occurrence of the arrhythmia after the ablation procedure.

When ablating tissue by means of electroporation, irrigation of the ablation electrode may or may not be necessary depending on the amount of tissue heating that results from administration of the short duration high energy pulse. The use of the SMT catheter for irreversible electroporation will require less energy and result in less tissue heating than when conventional catheters are employed. As a result, less or no irrigation may be required with the SMT catheter, the tissue temperature can be accurately monitored, and a temperature control mode of operation can be utilized to further reduce the risk of tissue charring, steam pops, and blood coagulation.

Therefore, in one embodiment for producing high quality lesions and for reducing the risk of charring, steam pops and blood coagulation, the energy delivered to the electrode is configured to cause irreversible electroporation of the tissue. In one embodiment this energy is configured to be in the form of pulses wherein the majority of the energy in the pulse is contained in a window of less than 20 milliseconds. In another embodiment the amplitude of the pulses are between 250 and 2500 volts.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” In addition, use of the term “based on,” above and in the claims is intended to mean, “based at least in part on,” such that an unrecited feature or element is also permissible.

The subject matter described herein can be embodied in systems, apparatus, methods, and/or articles depending on the desired configuration. The implementations set forth throughout do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and subcombinations of the disclosed features and/or combinations and subcombinations of several further features disclosed above. In addition, the logic flows depicted in the accompanying figures and/or described herein do not necessarily require the particular order shown, or sequential order, to achieve desirable results. Other implementations may be within the scope of the following claims.

What is claimed is:

1. A method for improving ablation lesion quality in cardiovascular tissue, comprising:
introducing into the body a catheter which includes an electrode which is mounted on a distal end of the catheter;
positioning the electrode to be in contact with the tissue to be ablated such that at least 65 percent of an exposed distal surface area of the electrode is in contact with the tissue to be ablated; and
applying electrical energy to the electrode sufficient to create an ablation lesion in the tissue.

2. The method of claim 1 wherein the electrical energy is radiofrequency energy.

3. The method of claim 1 wherein the electrical energy is comprised of one or more short duration, high voltage, pulses suitable for ablating tissue by means of irreversible electroporation.

4. The method of claim 1 wherein the catheter contains an irrigation channel.

5. The method of claim 4 further comprising delivering fluid through the irrigation channel;

6. The method of claim 5, wherein irrigation fluid passes through a plurality of channels located within the electrode.

7. The method of claim 1, further comprising monitoring a temperature of the electrode.

8. The method of claim 1, further comprising expanding transversely to the shaft of the catheter a plurality of deployable wings on the distal end of the catheter.

9. The method of claim 2, wherein the radiofrequency energy applied is between about 4 watts and about 15 watts and is applied for a period of 30 seconds or more.

10. The method of claim 2 wherein the radiofrequency energy is greater than or equal to 20 watts and is applied for a period less than or equal to 10 seconds.

11. The method of claim 5, wherein the irrigation fluid is delivered at an irrigation rate less than or equal to six milliliters per minute.

12. The method of claim 1, wherein at least fifty percent of the energy that passes out of the catheter through the electrode during ablation of the cardiovascular tissue passes through the tissue to be ablated.

13. The method of claim 3 wherein the majority of the energy in the pulse is contained in a window of less than 20 milliseconds.

14. The method of claim 3 wherein the amplitude of the pulse is between 250 and 2500 volts.

15. A method of ablating cardiovascular tissue to reduce the risk of tissue charring, steam pops and blood coagulation comprising:
introducing into the body a catheter which includes an irrigation channel and an electrode which is mounted on a distal end of the catheter;
positioning the electrode to be in contact with the tissue to be ablated;
delivering fluid through the irrigation channel at a rate of less than or equal to 6 milliliters/minute;
utilizing temperature control mode to control the delivery of energy to the electrode; and
creating an ablation lesion in the tissue.

16. The method of claim 15 in which at least 65 percent of an exposed distal surface area of the electrode is in contact with the tissue to be ablated.

17. The method of claim 15 in which a thermocouple located less than 500 microns from the surface of the ablation electrode is used to monitor the temperature.

18. The method of claim 15 in which the energy is radiofrequency energy.

19. The method of claim 15 in which the energy is configured to cause irreversible electroporation of the tissue.

20. The method of claim 15 further comprising expanding transversely to the shaft of the catheter a plurality of deployable wings located on the distal end of the catheter.
